(19) United States
(12) Patent Application Publication
(10) Pub. No.: US 2011/0259349 A1
(43) Pub. Date: Oct. 27, 2011

(54) DISPOSABLE ABSORBENT ARTICLE

(76) Inventor: Tina McDonnell, Frontenac, MO (US)

(21) Appl. No.: 13/091,644

(22) Filed: Apr. 21, 2011

Related U.S. Application Data
(60) Provisional application No. 61/326,500, filed on Apr. 21, 2010.

(51) Int. Cl. A61F 13/58; A61F 13/60; A61L 15/20

(52) U.S. Cl. 128/889; 604/385.03; 604/377; 604/385.05

(57) ABSTRACT

The invention relates to a disposable absorbent article used to absorb body fluids and to prevent skin breakdown and chafing under the breast. The article is secured to the front inner and outer lower part of the bra. The article can also be used under the abdominal skin fold to prevent skin to skin contact or to prevent chafing.

This application claims the benefit of U.S. Provisional Application No. 61/326,500, filed Apr. 21, 2010, the contents of which are incorporated herein by reference.

The invention relates to a disposable absorbent article used to absorb body fluids and to prevent skin breakdown and chafing under the breast. The article is secured to the front inner and outer lower part of the bra. The article can also be used under the abdominal skin fold to prevent skin to skin contact or to prevent chafing.

The rubbing together of skin can cause skin breakdown. When the skin breaks down, the tissues underneath it can become infected. This may lead to blood poisoning. Breaking of the skin can also include pressure sores, which can take a long time to heal. Prevention of the rubbing, as well as keeping the skin dry, helps eliminate skin breakdown.

Accordingly, a primary embodiment of the subject invention is to provide a multi-purpose, disposable, absorbent article which includes an adhesive strip that prevents the article from shifting or moving out of its desired position after placement, said article comprising an absorbent layer and a waterproof layer.

In further embodiments, said absorbent layer is directly bonded to said waterproof liner.

In another embodiment, said absorbent layer is made of cotton.

In another embodiment, said waterproof layer is made of plastic.

In further embodiments, said adhesive strips are pressure sensitive.

In yet another embodiment, said adhesive strips are covered by a peel-away layer that is removed prior to use.

Another embodiment of the subject invention is to provide an absorbent article that includes an absorbent layer and adhesive strips attached to a paper liner, each having pre-scored fold portions making the article size adjustable.

Still a further embodiment of the subject invention is to provide absorbent articles that are easy to manufacture in various sizes.

In a further embodiment, the article is a bra liner.

In another embodiment, the article is an abdominal liner and is placed under the abdominal fold.

These embodiments are attained by providing disposable absorbent articles, comprising an absorbent layer and an adhesive strip on opposite sides of a plastic liner. The absorbent material is attached to a liner to provide shape and support.

In a further embodiment, the absorbent layer is made of cotton with a thin layer of paper covering it.

In another embodiment, the article is about 6″ by 20″.

Further, methods are provided to prevent skin breakdown, said method comprising placement of the absorbent article between folds of skin to prevent rubbing together of said skin, and keeping said skin dry and free of breakdown.

### BRIEF DESCRIPTION OF THE DRAWINGS

FIG. 1 is a bottom view of the adsorbent article showing the fold and adhesive strips.

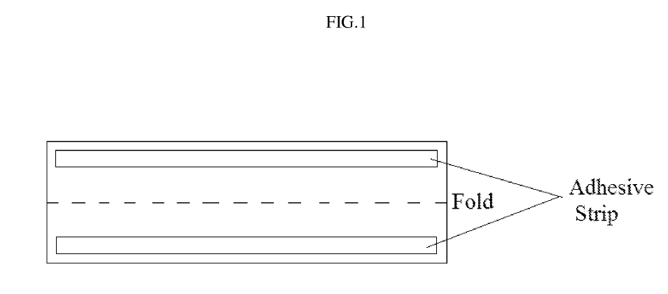

The absorbent article, as seen in FIG. 1, has a crease or fold in the middle and running the entire 20 inch length of the article. Adhesive strips are located on each side of the fold and are covered by a thin layer of removable paper.

The article may be configured for a variety of uses. It is pre-scored and easily pliable, and thus may be folded in any direction to form a desired shape. When the article is folded and the adhesive strips affixed to one another, a multi-purpose article with multiple absorbent layers results. Further, the article may be affixed directly to the patient providing an absorbent layer that is firmly held in place.

The article may be secured to each side (inner and outer) sides of the lower part of the bra. The fold of the article is placed on the bottom of the bra with each side of the article folded up to the inner and outer part of the bra. These sides of the article are secured to the bra by the adhesive strips located on the article. Perspiration could be wicked away, and the device would provide additional protection when folded in front of the bra. However, the overall thickness of the device should not affect the contour of the bra.

When the article is used as an abdominal liner the article is folded in half at the score and the two adhesive strips are secured to each other making the article 3 inches wide. The article is then placed under the abdominal fold. The device being folded would provide multiple layers of absorption and be superior to using solely an absorbent material. In addition, the device length would provide greater stability and would not move as easily as solely an absorbent material.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A multi-purpose, disposable absorbent article to be worn by a patient comprising
an absorbent layer covered in a thin layer of paper;
a waterproof liner; and
adhesive strips affixed to the opposite side of said liner as said absorbent layer.

2. The absorbent article as recited in claim 1, wherein said article is pre-scored for easy folding.

3. The absorbent article as recited in claim 1, wherein said absorbent layer is directly bonded to said waterproof liner.

4. The absorbent article as recited in claim 1, wherein said absorbent layer is made of cotton.

5. The absorbent article as recited in claim 1, wherein said waterproof liner is made of plastic.

6. The absorbent article as recited in claim 1, wherein said adhesive strips are pressure sensitive.

7. The absorbent article as recited in claim 1, wherein said adhesive strips are covered by a peel-away layer that is removed prior to use.

8. The absorbent article as recited in claim 1, wherein said article is a bra liner.

9. The absorbent article as recited in claim 1, wherein said article is an abdominal liner.

10. A method of preventing skin breakdown, said method comprising placement of the absorbent article as recited in claim 1 between folds of skin to prevent rubbing together of said skin, and keeping said skin dry and free of breakdown.
